(19) United States
(12) Patent Application Publication
(10) Pub. No.: US 2014/0277323 A1
(43) Pub. Date: Sep. 18, 2014

(54) ORAL NEURAL STIMULATOR

(71) Applicant: ORALTONE LLC, Boise, ID (US)

(72) Inventor: TERRELL F. TINGEY, EAGLE, ID (US)

(73) Assignee: ORALTONE LLC, Boise, ID (US)

(21) Appl. No.: 13/844,575

(22) Filed: Mar. 15, 2013

(51) Int. Cl. A61N 1/05

(52) U.S. Cl. CPC A61N 1/0548
USPC 607/134

(57) ABSTRACT

The present disclosure is directed to treatment of a migraine headache or other nervous system malady such as seizures and epilepsy by applying electrical stimulation to a subject's oral mucosa. Noninvasive electrodes may be placed against the subject's oral mucosa tissue by inserting a mouthpiece comprising the electrodes into the subject's mouth. One or more electrical pulses may be applied to the electrodes to stimulate the subject's cranial nerves and thereby treat the malady.

### BACKGROUND

1. Technical Field

The present disclosure relates generally to treatment of migraine headaches or other central nervous system maladies such as seizures and epilepsy. More particularly, the disclosure relates to treatment of a central nervous system malady through electrical stimulation of a person's brain and/or cranial nerves.

2. Background

Electrical stimulation of the brain and cranial nerves has been shown to have some efficacy in the treatment of migraine headaches and other central nervous system disorders. Neurostimulation can be administered through surgically implanted electrodes or through cutaneous contact. However, implantation of a neurostimulator may be an invasive procedure. Typical cutaneous stimulator devices are less effective than desirable.

What is needed, therefore, is a noninvasive treatment that effectively stimulates the brain and/or cranial nerves to treat a nervous system malady.

### SUMMARY

In one embodiment, an oral neural stimulator is disclosed. The oral neural stimulator includes a pair of electrodes on a mouthpiece and a pulse generator. The pulse generator is adapted to provide an electric pulse to the pair of electrodes. The mouthpiece is adapted to position the electrodes in contact with a subject's oral mucosa.

In another embodiment, a method of treating a nervous system malady is disclosed. The method includes inserting at least two electrodes into a subject's mouth, placing the electrodes against the subject's oral mucosa, and applying an electrical pulse to the electrodes.

In another embodiment, a method of treating a nervous system malady is disclosed. The method includes locating a pair of electrodes at a subject's oral mucosa and electrically stimulating the subject's trigeminal nerve by transmitting a voltage to the electrodes.

The present disclosure will now be described more fully with reference to the accompanying drawings, which are intended to be read in conjunction with both this summary, the detailed description, and any preferred or particular embodiments specifically discussed or otherwise disclosed. This disclosure may, however, be embodied in many different forms and should not be construed as limited to the embodiments set forth herein; rather, these embodiments are provided by way of illustration only so that this disclosure will be thorough, and fully convey the full scope of the disclosure to those skilled in the art.

### BRIEF DESCRIPTION OF THE DRAWINGS

Non-limiting and non-exhaustive embodiments of the present disclosure are described with reference to the following figures, wherein like reference numerals refer to like parts throughout the various views unless otherwise specified.

FIG. 1 depicts an oral neural stimulator according to an embodiment of the present disclosure;

FIG. 2 depicts a method of using an oral neural stimulator according to embodiments of the present disclosure;

FIG. 3 depicts a detachable mouthpiece of an oral neural stimulator according to embodiments of the present disclosure; and

FIG. 4 is a block diagram illustrating components of an oral neural stimulator according to an embodiment of the present disclosure.

### DETAILED DESCRIPTION

The present disclosure is directed to treatment of migraine headaches and other central nervous system maladies such as seizures and epilepsy by electrical stimulation to a subject's oral mucosa. In the following description, reference is made to the accompanying drawings that form a part thereof, and in which is shown by way of illustration specific exemplary embodiments in which the embodiments of the disclosure may be practiced. These embodiments are described in sufficient detail to enable those skilled in the art to practice the disclosure, and it is to be understood that modifications to the various disclosed embodiments may be made, and other embodiments may be utilized, without departing from the spirit and scope of the disclosure. The following detailed description is, therefore, not to be taken in a limiting sense.

Reference throughout this specification to “one embodiment,” “an embodiment,” “one example,” or “an example” means that a particular feature, structure, or characteristic described in connection with the embodiment or example is included in at least one embodiment of the present disclosure. Thus, appearances of the phrases “in one embodiment,” “in an embodiment,” “one example,” or “an example” in various places throughout this specification are not necessarily all referring to the same embodiment or example. Furthermore, the particular features, structures, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Embodiments in accordance with the present disclosure may be embodied as an apparatus, method, or computer program product. Accordingly, the present disclosure may take the form of an entirely hardware-comprised embodiment, an entirely software-comprised embodiment (including firmware, resident software, micro-code, etc.), or an embodiment combining software and hardware aspects that may all generally be referred to herein as a “circuit,” “module,” or “system.” Furthermore, embodiments of the present disclosure may take the form of a computer program product embodied in any tangible medium of expression having computer-usable program code embodied in the medium.

The block diagram in the attached figures illustrates the architecture and functionality of possible implementations of systems according to various embodiments of the present disclosure. In this regard, each block in the block diagram may represent a module, segment, or portion of code, which comprises one or more executable instructions for implementing the specified logical function(s). Each block and/or combination of blocks in the block diagram may be implemented by special purpose hardware-based systems or combinations of special purpose hardware and computer instructions that perform the specified functions or acts. These computer program instructions may also be stored in a computer-readable medium that can direct a computer or other programmable data processing apparatus to function in a particular manner, such that the instructions stored in the computer-readable medium implement the functions or acts specified in the block diagram block or blocks.

Embodiments of the present disclosure comprise a device designed to deliver electrical stimulation to a subject's oral mucosa to treat migraines or other central nervous system maladies such as seizures and epilepsy. Referring now to FIG. 1, the oral neural stimulator 100 according to embodiments of the present disclosure comprises electrodes 110, mouthpiece 120, and handle 130.

According to embodiments of the present disclosure, electrodes 110 comprise electrically conductive elements adapted to be placed in contact with a subject's biological tissue and transmit an electrical current to the tissue. In particular, embodiments comprise electrodes 110 adapted to be placed in contact with a subject's oral mucosa. Electrodes 110 may comprise any conductive material. Embodiments of the present disclosure comprise electrodes 110 manufactured from stainless steel. Other embodiments comprise electrodes 110 manufactured from or plated with 24-karat gold. In alternative embodiments, electrodes 110 comprise any material that conducts electricity. Electrodes 110 may preferably be manufactured from a corrosion-resistant material.

Mouthpiece 120 comprises extensions that act as a lip retractor and electrode 110 positioner. As depicted in FIG. 2, mouthpiece 120 comprises concave surface 122 having bifurcated arms 124 where electrodes 110 are placed. In embodiments of the present disclosure, mouthpiece 120 is adapted to conform to a subject's upper maxillary labial vestibule anterior alveolus 210 and position electrodes 110 thereat for contact with the subject's oral mucosa. In embodiments of the present disclosure, electrodes 110 are placed approximately one inch apart on mouthpiece 120. In alternative embodiments, electrodes 110 are selectively positioned to be placed in contact with a subject's oral mucosa. In embodiments, mouthpiece 120 comprises a thermoset plastic or other nonconductive, rigid material. Embedded wires 170 within mouthpiece 120 provide electrical communication between electrodes 110 and control circuitry.

Handle 130 is adapted to provide a holding and gripping surface for oral neural stimulator 100. In embodiments, handle 130 comprises a housing to contain control circuitry and/or power source to power the device 100. Referring now to FIG. 1, embodiments of handle 130 further comprise user control input 140, use indicator 150, and power button 160. In an embodiment of the present disclosure, user control input 140 comprises a slider switch by which a user may input a variable selection. Control input 140 may be used to set the intensity of the electric signal output at electrodes 110.

Referring now to FIG. 3, embodiments of the present disclosure comprise mouthpiece 120 that is detachable from handle 130. Accordingly, mouthpiece 120 may be removed for disposal or disinfection between uses with a reusable handle 130. Embodiments of mouthpiece 120 comprise embedded conductive wires 170 (depicted in FIG. 1) that lead to contacts 310 for electrical communication between control circuitry and electrodes 110.

Referring now to FIG. 4, embodiments of oral neural stimulator 100 comprise control module 410, pulse generator 420, electrodes 110, user control input 140, indicator 150, power button 160, and power source 430. In embodiments, control module 410, pulse generator 420, and power source 430 are housed within handle 130. Control module 410 comprises circuitry and/or software representing computer-readable instructions that direct pulse generator 420 to transmit electrical pulses to electrodes 110 according to user inputs. Pulse generator 420 comprises hardware adapted to generate electric waveform signals as directed by control module 410.

Control module 410 is adapted to receive inputs from user control input 140 and power button 160. In embodiments, control input 140 comprises a button slider that may be adjusted by a user. In response to adjustments made by a user to control input 140, control module 410 can direct pulse generator 420 to increase or decrease output voltage accordingly. In alternative embodiments, adjustments to control input 140 can cause control module 410 to direct pulse generator to increase or decrease the pulse width of the signal waveform accordingly. In embodiments, control input 140 comprises an analog or digital potentiometer. However, other implementations of user control input 140 fall under the scope of the present disclosure.

Power button 160 comprises a button switch that allows a user to turn oral neural stimulator 400 on or off. In embodiments, power button 160 comprises a switch adapted to connect or disconnect power source 430 to control module 410. In alternative embodiments, power button 160 acts to transmit an activation signal to control module 410 to begin or terminate electrical stimulation at electrodes 110.

Embodiments of the present disclosure comprise indicator 150. Indicator 150 comprises a visual signal to a user that oral neural stimulator 100 is currently outputting a current. In embodiments, indicator 150 comprises a monochromatic light-emitting diode (“LED”). In alternative embodiments, indicator 150 comprises a multi-colored LED, each color signaling a device status. For example, indicator 150 may output a green light if device 100 is working properly and a yellow light if the battery is running low. Alternative embodiments comprise a display embedded in handle 130 that provides for user inputs and/or information outputs.

In embodiments, power source 430 comprises a battery pack. The capacity of the battery pack may be selectively sized to fit anticipated power needs according to the present disclosure. In embodiments of the present disclosure, power source 430 comprises a receptacle for holding one or more replaceable batteries of standard size and type. In embodiments, power source 430 comprises a rechargeable battery pack. In alternative embodiments, power source 430 comprises an external electrical power supply such as an alternating current electrical source.

In operation, oral neural stimulator 100 may be used to treat migraine headaches or other central nervous system maladies such as seizures and epilepsy. Oral neural stimulator 100 can be utilized when a subject notices the symptoms or warning of a neural event such as aura, blurred vision, or pain. Oral neural stimulator 100 can be applied by inserting bifurcated arms 124 of mouthpiece 120 under the subject's upper lip. Due to the shape of mouthpiece 120, the subject's lips may be retracted as the mouthpiece 120 is inserted into the subject's mouth. Once mouthpiece 120 is in place in the subject's mouth, the electrodes 110 may be lightly pressed against the subject's upper maxillary labial vestibule anterior alveolus 210. Oral neural stimulator 110 may be used without applying conductive gel to the subject's tissue because electrical current may be carried by the oral mucosa. Embodiments of the present disclosure may function by stimulating the subject's maxillary nerve or other branches of the subject's trigeminal nerve through electrical pulses. In alternative embodiments, electrodes 110 are positioned against other oral mucosa surfaces of the subject.

A user can activate the device by pressing power button 160. In response, control module 410 transmits an activation signal to pulse generator 420, causing pulse generator 420 to generate an electrical signal to electrodes 110 via embedded wires 170. In embodiments, the electrical signal comprises a square waveform signal or an approximation of a square wave signal having an approximate pulse duration of five milliseconds and a frequency of approximately eight hertz. In embodiments, the generated pulses have a voltage up to approximately 15 volts. In alternative embodiments, various voltage levels, frequencies, duty cycles, and signal waveforms may be applied and still fall under the present disclosure.

The duty cycle and/or voltage level of the generated pulse may be altered by adjusting control input 140 to optimize the subject's comfort level and efficacy of treatment. Generally, an increased pulse width or increased voltage may increase the stimulation intensity felt by the subject.

Whenever the subject feels an onset of symptoms, oral neural stimulator 100 may be used for up to several minutes or until the symptoms are mitigated or eliminated. Oral neural stimulator 100 may be operated by the subject or a medical professional treating the subject. In embodiments, mouthpiece 120 may be removed from handle 130 after use for disposal or disinfection prior to any subsequent use.

Although the present disclosure is described in terms of certain preferred embodiments, other embodiments will be apparent to those of ordinary skill in the art, given the benefit of this disclosure, including embodiments that do not provide all of the benefits and features set forth herein, which are also within the scope of this disclosure. It is to be understood that other embodiments may be utilized, without departing from the spirit and scope of the present disclosure.

What is claimed is:

1. An oral neural stimulator comprising:
a pair of electrodes on a mouthpiece; and
a pulse generator adapted to provide an electric pulse to the pair of electrodes;
wherein the mouthpiece is adapted to position the pair of electrodes in contact with a subject's oral mucosa.

2. The oral neural stimulator of claim 1, wherein the mouthpiece further comprises a lip retractor.

3. The oral neural stimulator of claim 1, further comprising a handle, wherein the mouthpiece is detachable from the handle.

4. The oral neural stimulator of claim 1, wherein the mouthpiece is adapted to position the pair of electrodes against a subject's upper maxillary labial vestibule anterior alveolus.

5. The oral neural stimulator of claim 1, wherein the electrical pulse comprises a duration of approximately 5 milliseconds.

6. The oral neural stimulator of claim 1, wherein the electrical pulse comprises an approximation of a square waveform.

7. The oral neural stimulator of claim 1, wherein the electrical pulse comprises a voltage between approximately 10 and 20 volts.

8. The oral neural stimulator of claim 1, wherein the electrical pulse comprises a frequency between approximately 6 and 10 hertz.

9. A method of treating a nervous system malady comprising:
inserting at least two electrodes into a subject's mouth;
placing the at least two electrodes against the subject's oral mucosa; and
applying an electrical pulse to the at least two electrodes.

10. The method of claim 9, wherein placing the at least two electrodes against the subject's oral mucosa comprises setting the at least two electrodes against the subject's upper maxillary labial vestibule anterior alveolus.

11. The method of claim 9, further comprising stimulating the subject's trigeminal nerve.

12. The method of claim 11, further comprising stimulating the subject's maxillary nerve.

13. The method of claim 9, wherein the electrical pulse comprises a duration of approximately 5 milliseconds.

14. The method of claim 9, wherein the electrical pulse comprises an approximation of a square waveform.

15. The method of claim 9, wherein the electrical pulse comprises a voltage between approximately 10 and 20 volts.

16. The method of claim 9, wherein the electrical pulse comprises a frequency between approximately 6 and 10 hertz.

17. The method of claim 9, wherein the central nervous system malady comprises a migraine headache.

18. A method of treating a central nervous system malady, comprising:
locating a pair of electrodes at a subject's oral mucosa and
electrically stimulating the subject's trigeminal nerve by transmitting a voltage to the pair of electrodes.

19. The method of claim 18, wherein. electrically stimulating the subject's trigeminal nerve by transmitting a voltage to the pair of electrodes comprises generating a square waveform.

20. The method of claim 18, wherein the central nervous system malady comprises a migraine headache.
